(19) United States
(12) Patent Application Publication
(10) Pub. No.: US 2024/0197262 A1
(43) Pub. Date: Jun. 20, 2024

(54) Methods and Systems for Intramyocardial Tissue Displacement and Motion Measurement

(71) Applicant: University of Virginia Patent Foundation, Charlottesville, VA (US)

(72) Inventors: Frederick H. Epstein, Charlottesville, VA (US); Yu Wang, Charlottesville, VA (US); Changyu Sun, Columbia, MO (US)

(21) Appl. No.: 18/472,215

(22) Filed: Sep. 21, 2023

Related U.S. Application Data
(60) Provisional application No. 63/408,760, filed on Sep. 21, 2022.

(51) Int. Cl. A61B 5/00; G06V 10/44

(52) U.S. Cl. CPC A61B 5/7267; G06V 10/44

(57) ABSTRACT

An exemplary method and system are disclosed that employ deep learning neural-network(s) trained with displacement-encoded imaging data (i.e., DENSE data) to estimate intramyocardial motion from cine MRI images retrieved with balanced steady state free precession sequences (bFSSP) and other cardiac medical imaging modalities, including standard cardiac computer tomography (CT) images, magnetic resonance imaging (MRI) images, echocardiogram images, heart ultrasound images, among other medical imaging modalities described herein. The deep learning neural-network(s) can be trained using (i) contour motion data from displacement-encoded imaging magnitude data as inputs to the neural network and (ii) displacement maps derived from displacement-encoded imaging phase images for comparison to the outputs of the neural network for neural network adjustments during the training. The DENSE trained neural network can be used to calculate tissue displacement from bFSSP cine images.

### CROSS-REFERENCE TO RELATED APPLICATION

This application claims priority to and the benefit under 35 U.S.C § 119(e) of U.S. Provisional Patent Application Ser. No. 63/408,760, entitled “Method and System for Strain Analysis that Includes CMR-trained StrainNet to Echocardiography,” filed Sep. 21, 2022, which is hereby incorporated by reference herein in its entirety as if fully set forth below.

### GOVERNMENT LICENSE RIGHTS

This invention was made with government support under Grant No. HL147104 awarded by the National Institute of Health. The government has certain rights in the invention.

### TECHNICAL FIELD

This disclosure relates to the measurement of intramyocardial tissue displacement and motion from biomedical images using a neural network trained using displacement-encoded imaging measurements.

### BACKGROUND

Cardiac magnetic resonance (CMR) myocardial strain imaging can provide measurements of intramyocardial tissue displacement and motion that can be used to determine physical properties of the heart tissue such as strain, strain rate, torsion, twist, among others. These intramyocardial motions and strain measurements can beneficially be employed in the diagnostics, as well as prognostic, of various heart diseases such as cardiotoxicity due to anti-cancer therapy, coronary heart disease, heart valve problems, inflammatory conditions such as pericarditis, cardiac tumors, or scarring, and other damage from a heart attack.

CMR myocardial strain imaging techniques such as Displacement-ENcoding with Stimulated Echoes (DENSE) and other methods such as myocardial tagging can directly measure intramyocardial tissue displacement, but additional scans are needed, thereby lengthening the overall CMR examination time. In a DENSE acquisition, tissue displacement is encoded into the MRI phase images to allow for the quantification of intramyocardial displacement and the subsequent computation of myocardial strain. The technique is highly sensitive and accurate and is generally employed in research but has not been widely accepted for use in the clinical diagnostics of heart disease due to the associated cost of increased examination time.

To provide strain imaging or estimation of intramyocardial motion from standard cine MRI scans, post-processing techniques such as feature tracking (or “tissue tracking”) may be used that generally entails tracking myocardial borders in cine MRI videos. It, however, lacks the same degree of accuracy as compared to dedicated strain imaging techniques such as displacement-encoded imaging. Cine images are short movies that show heart motion throughout the cardiac cycle.

There is a benefit to improving the accuracy of displacement and strain analysis of standard cine MRI images and/or augmenting the measuring of intramyocardial motion in other cardiac medical imaging modalities.

### SUMMARY

In one embodiment, a computer implemented method measures intramyocardial tissue displacement with image data. The method includes retrieving a medical image of a subject with a magnetic resonance imaging (MRI) system, having at least one processor, wherein the MRI system retrieves the medical image with a balanced steady-state free precession (bSSFP) pulse sequence. The method determines, with the processor, intramyocardial tissue displacement within the medical image by using a neural network, wherein the neural network has been trained with training data calculated from Displacement-ENcoding with Stimulated Echoes (DENSE) image data.

An additional embodiment includes a system that implements the computer implemented method.

Yet another embodiment includes a non-transitory computer program product that stores instructions to implement the computer implemented method.

The trained neural network may be used to determine intramyocardial motion data, or a parameter derived therefrom (e.g., strain, strain rate, torsion, twist, activation time), for the diagnostics or treatment of cardiac disease or cardiac health-related conditions such as, but not limited to, coronary heart disease, heart valve problems, inflammatory conditions such as pericarditis, cardiac tumors, scarring and other damage from a heart attack, or for cardiac resynchronization therapy. DENSE images acquired of other parts of the body (of the head or brain) may be similarly used to train a neural network, e.g., for the diagnostics or treatment of brain-related disease or conditions such as, but not limited to, Parkinson's disease, tremor, epilepsy, neurodegenerative conditions or diseases, Alzheimer's, seizures, paralysis, or psychiatric disease.

Other aspects and features, according to the example embodiments of the disclosed technology, will become apparent to those of ordinary skill in the art upon reviewing the following detailed description in conjunction with the accompanying figures.

### BRIEF DESCRIPTION OF THE DRAWINGS

The patent or application file contains at least one drawing executed in color. Copies of this patent or patent application publication with the color drawing(s) will be provided by the Office upon request and payment of the necessary fee. Reference will now be made to the accompanying drawings, which are not necessarily drawn to scale.

FIG. 1 is a system diagram illustrating an operating environment capable of implementing aspects of the present disclosure of determining intramyocardial motion and/or measurand (e.g., strain, twist, and torsion) in medical image scans using a DENSE MRI training data set in accordance with an illustrative embodiment.

FIG. 2A shows an example method of using a DENSE-trained deep learning neural network for a given imaging modality in accordance with an illustrative embodiment.

FIG. 2B shows an example method of using a DENSE-trained deep learning neural network for a given imaging modality in accordance with an illustrative embodiment.

FIG. 3A shows a method of using a DENSE neural network in accordance with an illustrative embodiment.

FIG. 3B shows a method of training a DENSE neural network in accordance with an illustrative embodiment.

FIG. 3C shows another method of using a DENSE neural network in accordance with an illustrative embodiment.

FIG. 4A shows an example method of training a neural network using DENSE MRI training data set in a study in accordance with an illustrative embodiment.

FIG. 5 shows Table 1 of this disclosure.

FIG. 6A shows Table 2 of this disclosure.

FIG. 6B shows Table 3 of this disclosure.

FIG. 7 illustrates an example of a magnetic resonance imaging (MRI) system in accordance with an illustrative embodiment.

FIG. 8 is a computer architecture diagram showing a computing system capable of implementing aspects of the present disclosure in accordance with one or more embodiments described herein.

FIG. 9 is an illustration of training neural network with DENSE image data to measure tissue displacement from bSSFP cine image data.

FIG. 10 shows test population details of this disclosure.

FIG. 11 shows example end-systolic displacement and Ecc maps comparing StrainNet analysis of bSSFP cine MRI and DENSE for (A) a healthy volunteer (23-year-old woman) and (B) a patient (71-year-old woman) with heart failure with left bundle branch block. bSSFP=balanced steady-state free precession, DENSE=displacement encoding with stimulated echoes, Ecc=circumferential strain.

FIG. 12 shows example segmental circumferential strain-time curves for StrainNet analysis of bSSFP cine MRI, FT analysis of bSSFP cine MRI, and DENSE for (A) a healthy volunteer (28-year-old woman) and (B) a patient (42-year-old man) with myocardial infarction. The healthy volunteer has 30 frames with repetition time (TR) of 32.17 msec for bSSFP cine MRI and 24 frames with TR of 30 msec for cine DENSE; the patient has 25 frames with TR of 34.44 msec for bSSFP cine MRI and 29 frames with TR of 30 msec for cine DENSE. bSSFP=balanced steady-state free precession, DENSE=displacement encoding with stimulated echoes, Ecc=circumferential strain, FT=feature tracking.

FIG. 13 shows layer specific Ecc measurements for FT analysis of bSSFP cine MRI, StrainNet analysis of bSSFP cine MRI, and DENSE data.

FIG. 14 shows Correlation and Bland-Altman plots of StrainNet and FT analyses of apical cine bSSFP sections with DENSE for end-systolic (A) global and (B) segmental Ecc. bSSFP=balanced steady-state free precession, DENSE=displacement encoding with stimulated echoes, Ecc=circumferential strain, FT=feature tracking.

FIG. 15 shows a table of comparison data for tests according to this disclosure.

FIG. 16 shows additional Correlation and Bland-Altman plots of StrainNet and FT analyses of midventricular cine bSSFP sections with DENSE for end-systolic (A) global and (B) segmental Ecc. bSSFP=balanced steady-state free precession, DENSE=displacement encoding with stimulated echoes, Ecc=circumferential strain, FT=feature tracking.

FIG. 17 shows additional Correlation and Bland-Altman plots of StrainNet and FT analyses of midventricular cine bSSFP sections with DENSE for end-systolic (A) global and (B) segmental Ecc. bSSFP=balanced steady-state free precession, DENSE=displacement encoding with stimulated echoes, Ecc=circumferential strain, FT=feature tracking.

FIG. 18 shows bull's-eye plots of per-segment Ecc for intraclass correlation coefficient (ICC, %), coefficient of variation (CV), bias, and 95% limits of agreements comparing StrainNet and FT with DENSE. A linear mixed-effects model was performed over techniques for each segment, and an asterisk in the bias column indicates statistical significance with the reference, DENSE. DENSE=displacement encoding with stimulated echoes, Ecc=circumferential strain, FT=feature tracking.

### DETAILED DESCRIPTION

In some aspects, the disclosed technology relates to free-breathing parameter mapping with high-contrast image registration. Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

As discussed herein, a “subject” (or “patient”) may be any applicable human, animal, or other organisms, living or dead, or other biological or molecular structure or chemical environment, and may relate to particular components of the subject, for instance, specific organs, tissues, or fluids of a subject, may be in a particular location of the subject, referred to herein as an “area of interest” or a “region of interest.”

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [6] refers to the 6th reference in the list. All references cited and discussed in this specification are incorporated herein by reference in their entireties and to the same extent as if each reference was individually incorporated by reference.

In the following description, references are made to the accompanying drawings that form a part hereof and that show, by way of illustration, specific embodiments or examples. In referring to the drawings, like numerals represent like elements throughout the several figures.

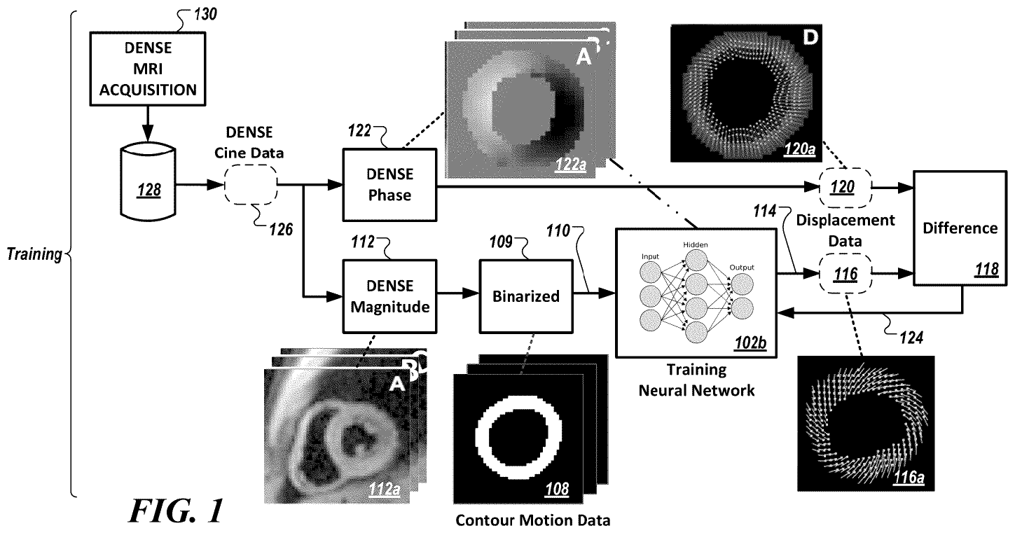

FIG. 1 is a system diagram illustrating an operating environment 100 capable of implementing aspects of the present disclosure of determining intramyocardial motion and/or measurand (e.g., strain, twist, torsion, activation time) in medical image scans using a DENSE MRI training data set in accordance with an illustrative embodiment. In the example shown in FIG. 1, the system comprising a trained neural network 102 (shown as 102a) is configured to generate, from provided biomedical images or videos 104 (shown as “Cine, MRI slice, Ultrasound, . . . , CT” 104), displacement-encoded data 106, e.g., a displacement map, that provides an estimation of intramyocardial motion in the provided biomedical images 104. The intramyocardial motion data 106 may be overlaid over the input biomedical images or videos 104. The intramyocardial motion data 106 can be analyzed, e.g., via a strain analysis 107, or other analyses, to determine strain, strain rate, torsion, twist, activation time, among others.

In the example shown in FIG. 1, the neural network 102 (shown as 102b) has been trained (i.e., configured) to have a correspondence between (i) the contour motion from an input displacement encoded image training data set (e.g., DENSE data set) and (ii) an output displacement encoded data indicating the intramyocardial motion of a subject. Examples of these biomedical images to which intramyocardial motion can be ascertained and overlaid include, and without being limiting, cardiac computer tomography (CT) images, magnetic resonance imaging (MRI) images, echocardiogram images, heart ultrasound images.

To create this correspondence between the contour motion data and the displacement data, the neural network 102b is trained using a sequence of contour motion data 108 as its input 110 in which the contour motion data 108 is derived from displacement encoded magnitude data 112 (shown as “DENSE magnitude” data 112). The contour motion data 108 is generated from a binary operator 109 configured to binarize as well as scale and/or crop the displacement encoded magnitude data 112. The neural network 102b outputs 114 displacement data 116 (shown as 116a) that are then compared (shown as “Difference” module 118) to the ground-truth displacement data 120 (shown as 120a) derived from displacement encoded phase data 122 (shown as “DENSE phase” data 122). The comparison (e.g., via a subtraction operator or a SoftMax operator) can generate feedback 124 to adjust the weights of the neural network 102b. Example images of the displacement encoded magnitude data 112 and displacement encoded phase data 122 are shown as 112a and 122a, respectively. The displacement encoded magnitude data (e.g., 112, 112a) and phase data (e.g., 122, 122a) can be generated from cine DENSE data 126 retrieved from a data store 128 and having been acquired through DENSE MRI acquisition 130, e.g., as described in U.S. Patent Publication no. 20190302210, which is incorporated by reference in its entirety.

### Example Method of Operation

The DENSE-trained deep learning neural network and associated method can operate with an imaging modality of any spatial resolution and any temporal frames.

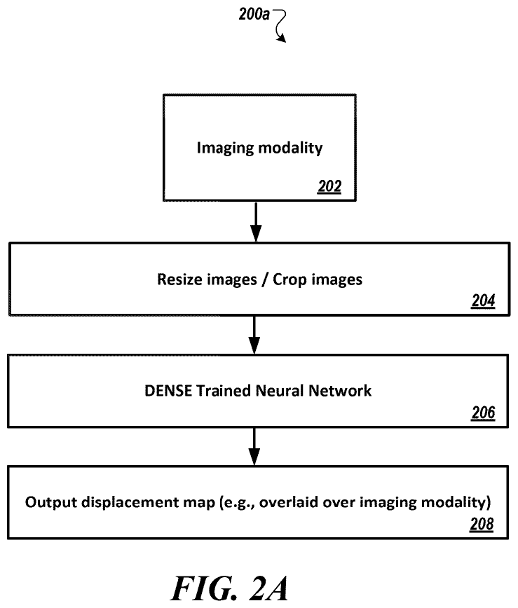

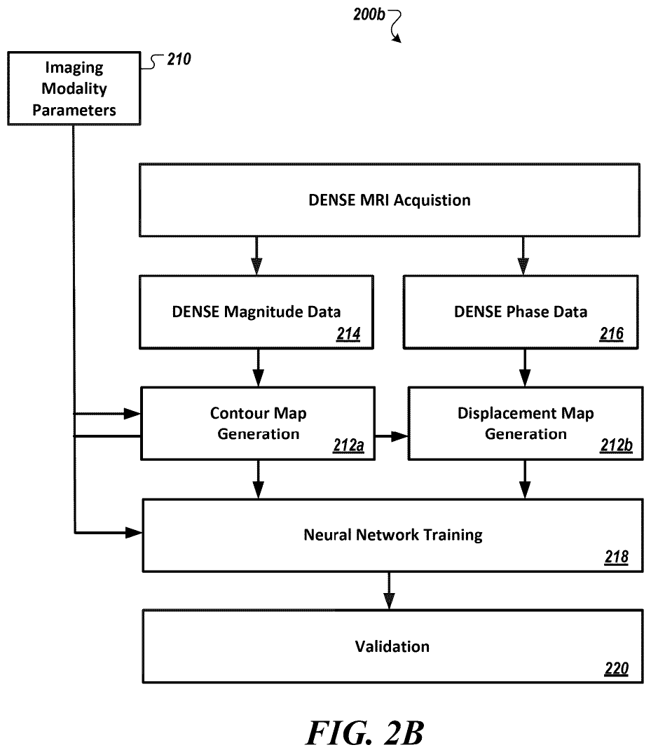

FIG. 2A shows an example method 200a of using a DENSE-trained deep learning neural network for a given imaging modality in accordance with an illustrative embodiment. In FIG. 2A, a set of time-series images are acquired 202 from an imaging modality such as cardiac computer tomography (CT) images, magnetic resonance imaging (MRI) images, echocardiogram images, or heart ultrasound images. The acquired images are resized (204) as pre-processed images to match the size of the trained data set of the trained neural network (e.g., 102a, 102b) and cropped to a region of interest. The pre-processed images provided (206) as input to the trained neural network can then provide a displacement map. The displacement map can be outputted (208) to be used for the diagnostics or the treatment of cardiac disease or the cardiac health-related conditions.

FIG. 2B shows an example method 200b of using a DENSE-trained deep learning neural network for a given imaging modality.

Conventional MRI technology discussed herein is discussed in 20190302210, which is incorporated by reference in its entirety as if set forth fully herein. Phase-contrast displacement encoding that has been used for myocardial imaging and cine DENSE (Displacement Encoding with Stimulated Echoes) imaging have emerged as a strain imaging technique that can offer high spatial resolution, equivalent accuracy, better reproducibility, and where strain analysis is less time-consuming. DENSE can also provide quantification of numerous features from myocardial tissue, where tissue displacement is encoded as a phase of a stimulated-echo signal (STE). In an embodiment, a method includes acquiring magnetic resonance data associated with physiological activity in an area of interest of a subject, where the acquired magnetic resonance data includes one or more phase-encoded data sets. The operation may include acquiring frames comprising a stimulated echo, a T1 relaxation echo, and a stimulated anti-echo. The method, configured in software, determines, from at least one or more phase-encoded data sets, an output data set (e.g., displacement-encoded information) corresponding to the physiological activity in the area of interest. Reconstruction of the images includes performing phase unwrapping of the phase-encoded data set using region growing along multiple pathways based on phase predictions.

MRI imaging encompasses techniques such as the acquisition of “cine images.” Cine images are short movies that can show heart motion throughout the cardiac cycle in short-axis. For example, measurement of left ventricular (LV) mass, ejection fraction, percentage of LV mass subtended by scar, and extracellular volume may be some of the heart tissue qualities studied with cine data. Cine DENSE, therefore, measures myocardial displacements by encoding tissue displacement into the signal phase. Displacement encoding frequencies (kₑ) are selected to balance signal-to-noise ratio, displacement sensitivity, and artifact suppression, resulting in phase wrapping during systole.

Spatio-temporal phase unwrapping is required to compute Lagrangian motion trajectories and strain. Phase unwrapping may be aided by delineating the myocardium using myocardial contours. Displacement encoding with stimulated echoes (DENSE), therefore, may be described as tools that measure myocardial displacements using the signal phase.

In FIG. 2B, the DENSE training data set are modified to match that of an imaging modality of interest. The parameters of an imaging modality are determined (210), such as size. The image parameters are used to adjust (shown as 212a, 212b) the image size of the training set of the contour data objects (e.g., 108) and the displacement data (e.g., 120). As previously discussed, the contour data object (e.g., 108) are generated (214) from DENSE magnitude data, and the displacement data (e.g., 120) are generated (216) from DENSE phase data. The neural network is trained (218) using the contour data (from step 212a) and displacement map (from step 212b). Following training, the neural network is validated (220). Once trained, the neural network can be used to determine intramyocardial tissue displacement and motion from other imaging modalities having a similar image size. The process of FIG. 2B can be used in combination with the process of FIG. 2A.

Per FIGS. 2A and 2B, while most methods of operations are limited to fixed image size or have to be cut to specific temporal frames, the exemplary method of use or training can handle the spatial resolution of various imaging modalities by resizing those images. And for the temporal frames, the exemplary method can accept any length of frames. This robust operation allows the exemplary method to be applied to a broader range of images.

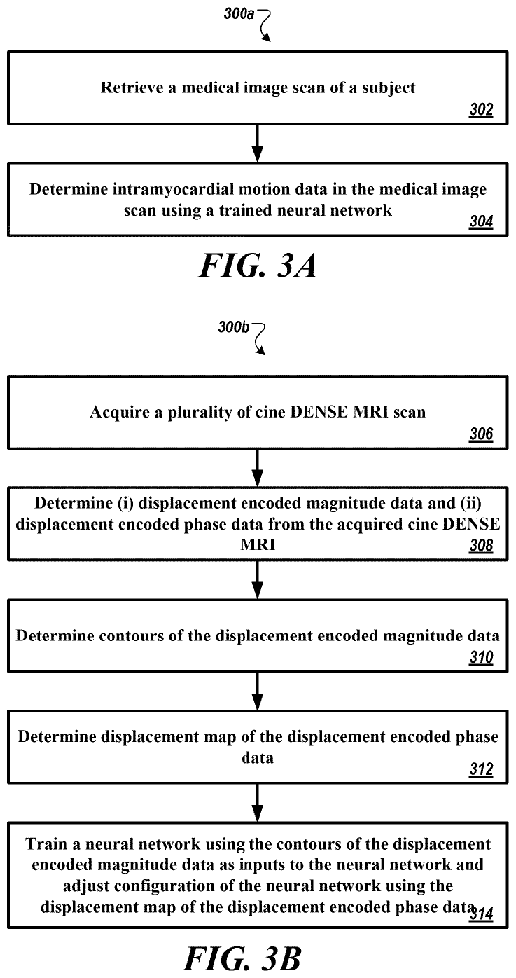

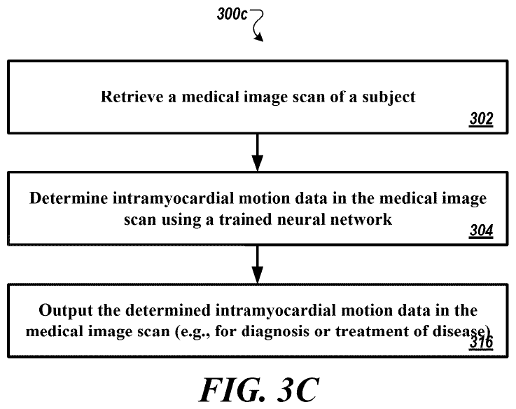

FIGS. 3A-3C each shows an example method of operation of using and/or training a DENSE neural network in accordance with an illustrative embodiment.

### Method of Using a DENSE Neural Network

FIG. 3A shows a method 300a of using a DENSE neural network in accordance with an illustrative embodiment. The method 300a includes retrieving (302) a medical image scan of a subject. A set of time-series images are acquired from an imaging modality such as cardiac computer tomography (CT) images, magnetic resonance imaging (MRI) images, echocardiogram images, or heart ultrasound images.

The method 300a then includes determining (304) intramyocardial motion data in the medical image scan using a trained neural network, e.g., trained according to the description of FIG. 1 or FIG. 3B.

### Method of Training a DENSE Neural Network

FIG. 3B shows a method 300b of training a DENSE neural network in accordance with an illustrative embodiment. The method 300b includes acquiring (306) a plurality of cine DENSE MRI scan. Example method of acquiring the DENSE MRI scan is described in U.S. Patent Publication no. 20190302210, which is incorporated by reference in its entirety.

The method 300b then includes determining (308) (i) displacement encoded magnitude data and (ii) displacement encoded phase data from the acquired cine DENSE MRI. Example method such determination is also provided in U.S. Patent Publication no. 20190302210 or US20200249306.

The method 300b then includes determining (310) contours (e.g., contour object data) of the displacement encoded magnitude data. In some embodiments, segmentation of LV myocardium may be performed semiautomatically using motion-guided segmentation, and manual correction was applied if needed. Example method of determining the contour data in the DENSE MRI scan is described in U.S. Patent Publication no. 20190302210.

The method 300b then includes determining (312) displacement map (e.g., displacement object data) of the displacement encoded phase data.

The method 300b then includes training (314) a neural network using the contour object data of the displacement encoded magnitude data as inputs to the neural network and adjusting configuration of the neural network using the displacement map object data of the displacement encoded phase data, e.g., as described in relation to FIG. 1. An example training may involve using multi-fold cross-validation. The neural network may be trained using an Adam optimizer configured to execute a pre-defined number of epochs (e.g., 100-500 epochs) or until training criteria are met. The training used the end-point-error based on cross-entropy or mean squared error, among others, as the loss function.

### Another Method of Using a DENSE Neural Network

FIG. 3C shows a method 300c of using a DENSE neural network in accordance with an illustrative embodiment. The method 300c includes retrieving (302) a medical image scan of a subject, e.g., as described in FIG. 3A. A set of time-series images are acquired from an imaging modality such as cardiac computer tomography (CT) images, magnetic resonance imaging (MRI) images, echocardiogram images, or heart ultrasound images.

The method 300c then includes determining (304) intramyocardial motion data in the medical image scan using a trained neural network, e.g., trained according to the description of FIG. 1 or FIG. 3B.

The method 300c then includes outputting (306) the determined intramyocardial motion data in the medical image scan (e.g., for diagnosis or treatment of disease). The output may include intramyocardial motion data for the diagnostics or treatment of cardiac disease or cardiac health-related conditions such as, but not limited to, coronary heart disease, heart valve problems, inflammatory conditions such as pericarditis, cardiac tumors, scarring, and other damage from a heart attack, or for cardiac resynchronization therapy. DENSE images acquired of other parts of the body (of the head or brain) may be similarly used to train a neural network, e.g., for the diagnostics or treatment of brain-related disease or conditions such as, but not limited to, Parkinson's disease, tremor, epilepsy, neurodegenerative conditions or diseases, Alzheimer's, seizures, paralysis, or psychiatric disease.

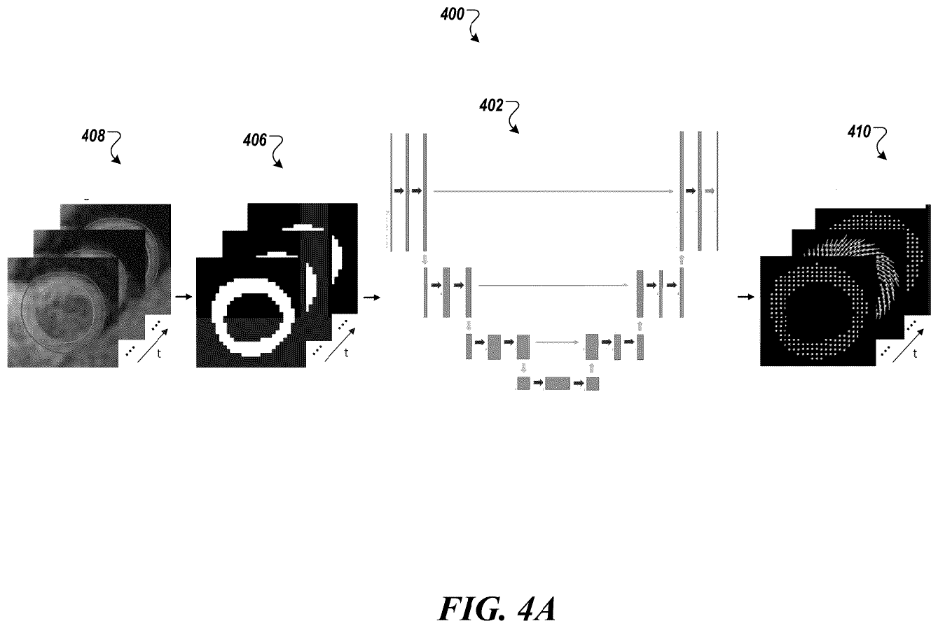

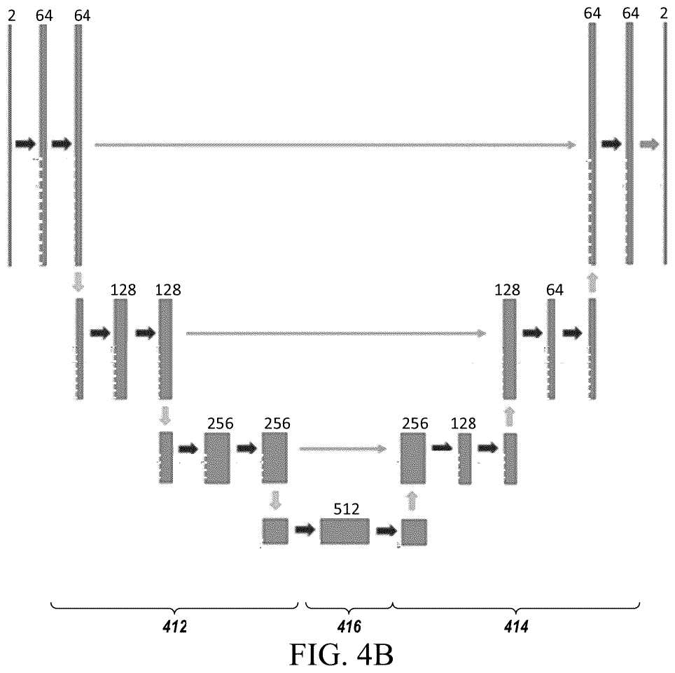

Various aspects of the disclosed technology may still be more fully understood from the following description of example implementations and corresponding results and the images of FIGS. 4A and 4B. Some experimental data are presented herein for purposes of illustration and should not be construed as limiting the scope of the disclosed technology in any way or excluding any alternative or additional embodiments.

A study was conducted to train a 3D-UNet neural network using DENSE MRI training data set to estimate intramyocardial motion from contour motion. Two workflows were implemented: a two-step FlowNet2-based framework with a through-time correction network and a 3D (2D+t) Unet framework. Both networks depicted cardiac contraction and abnormal motion patterns. The 3D Unet showed excellent reliability for global circumferential strain (Ecc) and good reliability for segmental Ecc, and it outperformed commercial FT for both global and segmental Ecc. FIG. 4A shows an example method of training a neural network using DENSE MRI training data set in a study in accordance with an illustrative embodiment.

Methods (3D Unet). In the study, the 3D-UNet neural network as a 3D convolutional neural network (CNN) 102b (in this figure shown as 402) with an encoder-decoder structure was configured and trained to estimate intramyocardial displacement 116 (shown as 404) from contour motion data objects 108 (shown as 406). The contour motion data objects 406 included a set of time series data having myocardial contour objects derived from DENSE magnitude images 112 (shown as 408). The ground truth data 120 (not shown) included data from DENSE tissue displacement measurements derived from DENSE phase images. Because the DENSE and cine images at matched slice locations share similar motion patterns, the study validated the trained neural network model using contour data derived from the standard cine images.

FIG. 4B shows example inputs of the 3D-Unet neural network 402. FIG. 4B show a sequence of myocardial contour objects derived from DENSE magnitude images 408. FIG. 4B also shows a sequence of the corresponding output intramyocardial displacement 404.

Data pre-processing: To prepare the DENSE and cine images for the training and validation operation, the training and validation data set comprising the left ventricular portion of DENSE and cine images were segmented by via binarization operation that fill the myocardium area with a value of “1” and the outside area and blood pool with a value of “0”. The images were cropped to a fixed size: Nₓ×N_{y} (e.g., 48*48 pixels to include the full left ventricle region). Data augmentation was performed using a 90° rotation. Cine images (validation data set) were scaled to match the resolution range of DENSE training images and also cropped into 48*48 pixels. Cine images were scaled to match the resolution range of DENSE images. The neural network is configured with an input size of Nₓ×N_{y}×Nₜ (e.g., 48*48*Nₜ), in which Nₜ represents the number of temporal frames and the output size is set as 2×Nₓ×N_{y}×Nₜ (e.g., 2*48*48*Nₜ) in which the factor of 2 accounts for the displacements in two directions.

Training. 5-fold cross-validation was applied to the training datasets. The model was trained with the Adam optimizer and a total of 300 epochs. The initial learning rate was set to 1e-4, with a halving schedule for each 100 epochs. The training used the end-point error as the loss function.

Datasets: The study trained the neural network using DENSE training datasets according to the table of FIG. 4A. The training data set included (i) data from 60 volunteers and (ii) data from 42 patients with various pathologies such as left bundle branch block (LBBB), hypertrophic cardiomyopathy, dilated cardiomyopathy, infarction, coronary artery disease and hypertension. The trained neural network model was validated on cine images of 10 volunteers and 18 patients in 3 short-axis views (base, mid-level, and apex). For the TC-FlowNet2 portion of the study, the datasets were divided into two parts to separately train DENSE-trained FlowNet2 and the correction network, thus the testing dataset number (15 subjects, 48 slices) was half the size as that used for the 3D Unet. Commercial feature-tracking (suiteHEART, Neosoft, WI) was also used to measure strain from cine images.

### DISCUSSION

Cardiac magnetic resonance (CMR) myocardial strain imaging (also referred to as “strain MRI”) is used diagnostically and prognostically for many types of heart disease. Feature tracking (FT) is a widely used and convenient method for strain MRI, as it applies post-processing algorithms directly to standard cine images to assess strain. It is, however, less accurate than strain-dedicated acquisitions like displacement encoding with stimulated echoes (DENSE), especially for segmental strain. FT methods track myocardial contours rather than intramyocardial tissue because the myocardium presents uniform signal on cine MRI, lacking features to track. The intramyocardial motion is then (imperfectly) estimated using optical-flow-based methods applied to the times series of endocardial and epicardial contours. Feature tracking is a method of measuring contour displacement by tracking points along images of the endocardial and epicardial contours. The tracking may occur on a frame by frame basis by identifying explicit features of the contours and then finding those features on later frames of images.

In contrast, DENSE directly measures intramyocardial tissue displacement; however, it requires additional acquisitions. As DENSE provides both myocardial contours and accurate intramyocardial tissue displacement information, this disclosure investigated the use of DENSE data to train deep networks to predict intramyocardial tissue motion from contour motion. This deep learning (DL) approach may provide the clinical convenience of FT and accuracy similar to DENSE.

A 3D Unet, trained using DENSE datasets to predict intramyocardial motion from contour motion, outperformed both TC-FlowNet2 and commercial FT for the measurement of both global and segmental E_{cc}, for which DENSE data at matched locations served the reference standard. The proposed DTSA network showed the ability to depict detailed intramyocardial motion, which is challenging for optical-flow-based FT methods. The proposed network showed improved performance for both global and segmental Ecc compared to commercial FT results.

An additional kind of image acquisition is referred to as balanced steady state free precession (bSSFP) and is much faster than DENSE or feature tracking procedures. Many MRI systems are equipped to gather bSSFP images with standard equipment across the industry of medical imaging, particularly in the form of movie-like imagery known as cine images. Balanced steady state free precession imagery is discussed at length in United States Patent No. 20180329007 (Guo et al.), which describes the kinds of RF pulse sequences used during magnetic resonance imaging to accomplish bSSFP images discussed herein. This disclosure makes advancements in the field of gathering medical images in standard bSSFP formats but being able to track intramyocardial movements with the accuracy of DENSE image processing.

### Example MRI System

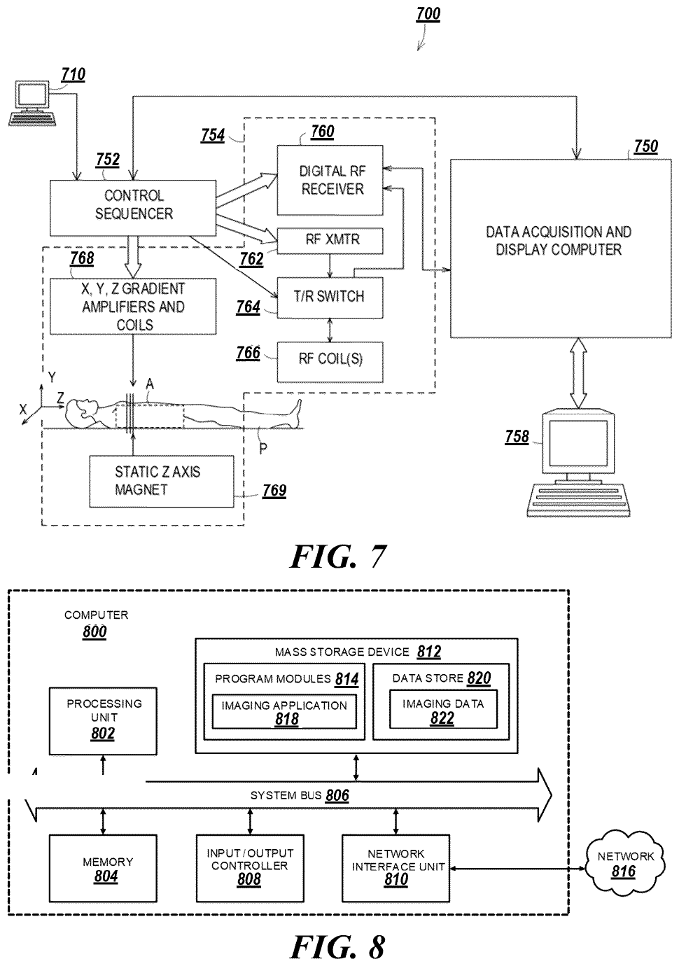

FIG. 7 illustrates an example of a magnetic resonance imaging (MRI) system 700, including a data acquisition and display computer 750 coupled to an operator console 710, an MRI real-time control sequencer 752, and an MRI subsystem 754. The MRI subsystem 754 may include XYZ magnetic gradient coils and associated amplifiers 768, a static Z-axis magnet 769, a digital RF transmitter 762, a digital RF receiver 760, a transmit/receive switch 764, and RF coil(s) 766. The MRI subsystem 754 may be controlled in real-time by control sequencer 752 to generate magnetic and radio frequency fields that stimulate magnetic resonance phenomena in a living subject, patient P, to be imaged. A contrast-enhanced image of an area of interest A of the patient P may be shown on display 758. The display 758 may be implemented through a variety of output interfaces, including a monitor, printer, or data storage.

The area of interest “A” corresponds to a region associated with one or more physiological activities in patient “P”. The area of interest shown in the example embodiment of FIG. 7 corresponds to a chest region of patient “P”, but the area of interest for purposes of implementing aspects of the disclosure presented herein is not limited to the chest area. It should be recognized and appreciated that the area of interest can be one or more of a brain region, heart region, and upper or lower limb regions of the patient “P”, for example. Physiological activities that may be analyzed by methods and systems in accordance with various embodiments of the present disclosure may include, but are not limited to, muscular movement or fluid flow in particular areas of interest.

It should be appreciated that any number and type of computer-based medical imaging systems or components, including various types of commercially available medical imaging systems and components, may be used to practice certain aspects of the present disclosure. Systems, as described herein with respect to example embodiments, are not intended to be specifically limited to magnetic resonance imaging (MRI) implementations or the particular system shown in FIG. 7.

One or more data acquisition or data collection steps as described herein in accordance with one or more embodiments may include acquiring, collecting, receiving, or otherwise obtaining data such as imaging data corresponding to an area of interest. By way of example, data acquisition or collection may include acquiring data via a data acquisition device, receiving data from an on-site or off-site data acquisition device or from another data collection, storage, or processing device. Similarly, data acquisition or data collection devices of a system in accordance with one or more embodiments of the present disclosure may include any device configured to acquire, collect, or otherwise obtain data, or to receive data from a data acquisition device within the system, an independent data acquisition device located on-site or off-site, or another data collection, storage, or processing device.

### Example Computing Environment and System

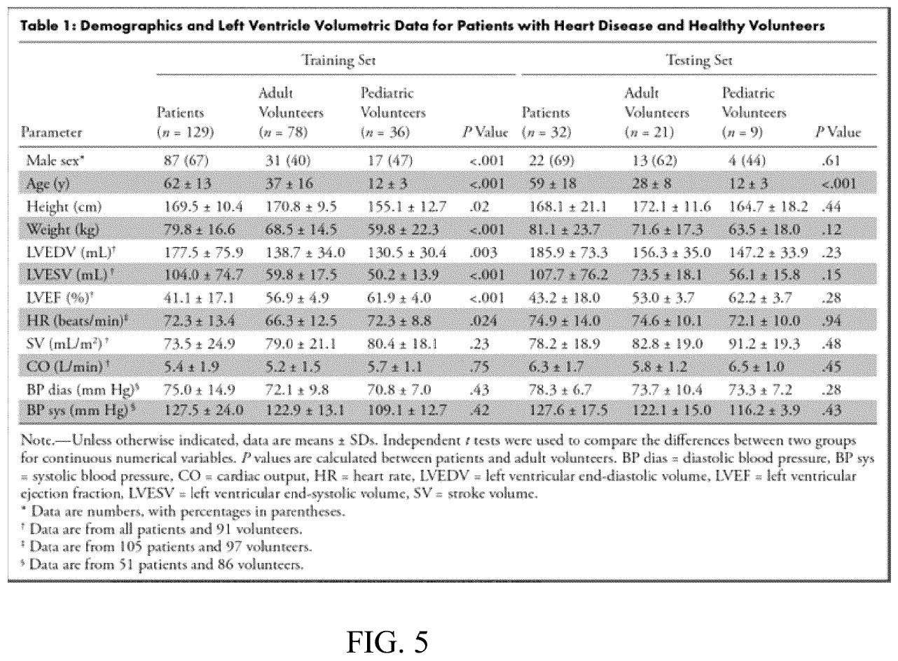

FIG. 8 is a computer architecture diagram showing a computing system capable of implementing aspects of the present disclosure in accordance with one or more embodiments described herein. A computer 800 may be configured to perform one or more functions associated with embodiments of intramyocardial motion and/or measurand analysis using a neural network illustrated in one or more of FIGS. 1-6. For example, the computer 800 may be configured to perform operations of the method shown in FIG. 1 and as described above. It should be appreciated that the computer 800 may be implemented within a single computing device or a computing system formed with multiple connected computing devices. The computer 800 may be configured to perform various distributed computing tasks, which may distribute processing and/or storage resources among the multiple devices. The data acquisition and display computer 150 and/or operator console 110 of the system shown in FIG. 1 may include one or more systems and components of the computer 800.

As shown, the computer 800 includes a processing unit 802 (“CPU”), a system memory 804, and a system bus 806 that couples the memory 804 to the CPU 802. The computer 800 further includes a mass storage device 812 for storing program modules 814. The program modules 814 may be operable to perform one or more functions associated with embodiments of the method as illustrated in one or more of FIGS. 1-6 discussed above, for example, to cause the computer 800 to perform operations of the intramyocardial motion and/or measurand analysis using a neural network shown in FIG. 1 and as described above. The program modules 814 may include an imaging application 818 for performing data acquisition functions as described herein, for example, to receive image data corresponding to magnetic resonance imaging of an area of interest. The computer 800 can include a data store 820 for storing data that may include imaging-related data 822 such as acquired image data, and a modeling data store 824 for storing image modeling data, or other various types of data utilized in practicing aspects of the present disclosure.

The mass storage device 812 is connected to the CPU 802 through a mass storage controller (not shown) connected to the bus 806. The mass storage device 812 and its associated computer-storage media provide non-volatile storage for the computer 800. Although the description of computer-storage media contained herein refers to a mass storage device, such as a hard disk or CD-ROM drive, it should be appreciated by those skilled in the art that computer-storage media can be any available computer storage media that can be accessed by the computer 800.

By way of example, and not limitation, computer-storage media (also referred to herein as a “computer-readable storage medium” or “computer-readable storage media”) may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid-state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 800. Transitory signals are not “computer-storage media”, “computer-readable storage medium” or “computer-readable storage media” as described herein.

According to various embodiments, the computer 800 may operate in a networked environment using connections to other local or remote computers through a network 816 via a network interface unit 810 connected to the bus 806. The network interface unit 810 may facilitate connection of the computing device inputs and outputs to one or more suitable networks and/or connections such as a local area network (LAN), a wide area network (WAN), the Internet, a cellular network, a radio frequency network, a Bluetooth-enabled network, a Wi-Fi enabled network, a satellite-based network, or other wired and/or wireless networks for communication with external devices and/or systems. The computer 800 may also include an input/output controller 808 for receiving and processing input from a number of input devices. Input devices may include one or more keyboards, mice, stylus, touchscreens, microphones, audio capturing devices, or image/video capture devices. An end-user may utilize such input devices to interact with a user interface, for example, a graphical user interface, for managing various functions performed by the computer 800.

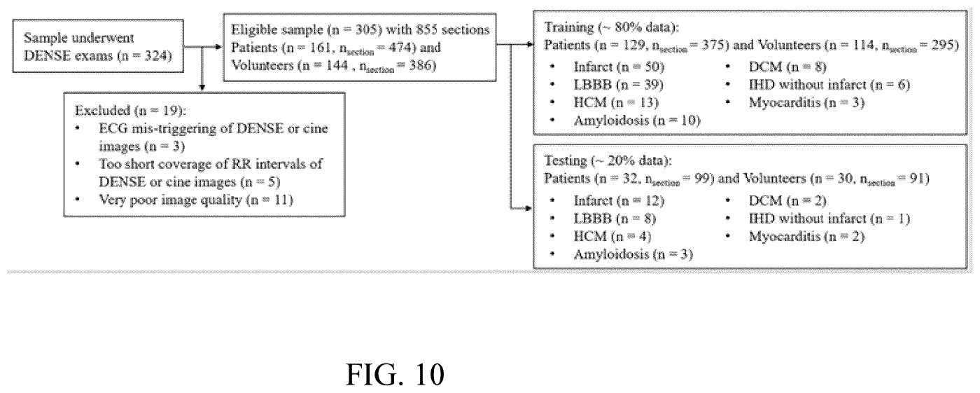

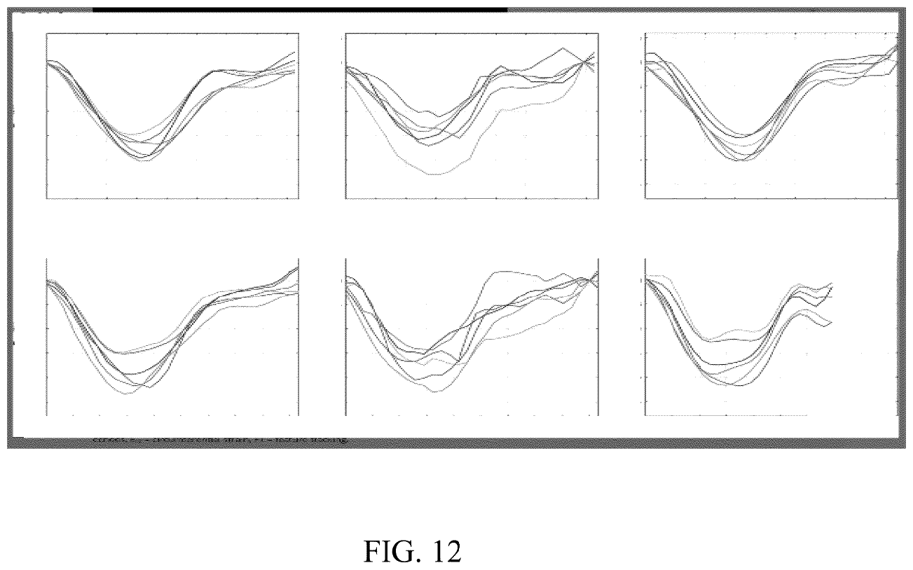

The bus 806 may enable the processing unit 802 to read code and/or data to/from the mass storage device 812 or other computer-storage media. The computer-storage media may represent apparatus in the form of storage elements that are implemented using any suitable technology, including but not limited to semiconductors, magnetic materials, optics, or the like. The computer-storage media may represent memory components, whether characterized as RAM, ROM, flash, or other types of technology. The computer-storage media may also represent secondary storage, whether implemented as hard drives or otherwise. Hard drive implementations may be characterized as solid state or may include rotating media storing magnetically-encoded information. The program modules 814, which include the imaging application 818, may include instructions that, when loaded into the processing unit 802 and executed, cause the computer 800 to provide functions associated with embodiments illustrated in FIGS. 3-14. The program modules 814 may also provide various tools or techniques by which the computer 800 may participate within the overall systems or operating environments using the components, flows, and data structures discussed throughout this description.

In general, the program modules 814 may, when loaded into the processing unit 802 and executed, transform the processing unit 802 and the overall computer 800 from a general-purpose computing system into a special-purpose computing system. The processing unit 802 may be constructed from any number of transistors or other discrete circuit elements, which may individually or collectively assume any number of states. More specifically, the processing unit 802 may operate as a finite-state machine, in response to executable instructions contained within the program modules 814. These computer-executable instructions may transform the processing unit 802 by specifying how the processing unit 802 transitions between states, thereby transforming the transistors or other discrete hardware elements constituting the processing unit 802.

Encoding the program modules 814 may also transform the physical structure of the computer-storage media. The specific transformation of physical structure may depend on various factors, in different implementations of this description. Examples of such factors may include but are not limited to the technology used to implement the computer-storage media, whether the computer storage media are characterized as primary or secondary storage, and the like. For example, if the computer-storage media are implemented as semiconductor-based memory, the program modules 814 may transform the physical state of the semiconductor memory, when the software is encoded therein. For example, the program modules 814 may transform the state of transistors, capacitors, or other discrete circuit elements constituting the semiconductor memory.

As another example, the computer-storage media may be implemented using magnetic or optical technology. In such implementations, the program modules 814 may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

This disclosure is particularly useful, though not limited to, acquiring medical images in a convenient and affordable format, such as bSSFP described above, and then estimate, calculate, and/or track tissue displacement and even strain within the myocardium with deep learning neural networks. In non-limiting embodiments, the neural networks are trained with DENSE data and then used with bSSFP image data as inputs.

The following abbreviations may be used in this disclosure:
bSSFP=balanced steady-state free precession,
DENSE=displacement encoding with stimulated echoes,
DL=deep learning,
Ecc=circumferential strain,
EPE=end-point error,
FT=feature tracking,
ICC=intraclass correlation coefficient,
LV=left ventricle,
3D=three-dimensional,
2D=two-dimensional.

This disclosure refers to computer implemented software, computerized methods, and non-transitory computer program products with the industry term, StrainNet, but that term is not limiting of the disclosure. Software may be implemented for the analysis of routine cardiac cine MRI provides clinical convenience similar to feature tracking and better agreement with displacement encoding with stimulated echoes for both global and segmental circumferential strain. In both healthy controls and patients with various heart diseases, the methods, system, and products of this disclosure (i.e., the disclosed software) predicted intra-myocardial displacement and strain from myocardial contour motion and showed good agreement with the displacement encoding with stimulated echoes (DENSE) technique (average end-point error, 0.75 mm+0.35). In fact, for the analysis of routine cine MRI, the disclosed software showed better agreement than commercial feature tracking with DENSE for both global and segmental circumferential strain, with intraclass correlation coefficients of 0.87 versus 0.72 and 0.75 versus 0.48, respectively.

Myocardial strain imaging using cardiac MRI has several applications, including the detection of chemotherapy induced cardiotoxicity (1), optimization of cardiac resynchronization therapy (2), outcome prediction after myocardial infarction (3), and identification of subclinical cardiac dysfunction in obesity and diabetes (4), cardiac MRI methods such as myocardial tagging (5), displacement encoding with stimulated echoes (DENSE) (6,7), and strain encoded imaging (8) acquire images specifically designed to measure intramyocardial deformation and strain and can be referred to as strain dedicated methods. Alternatively, feature tracking (FT) (9) estimates strain from routine cine balanced steady-state free precession (bSSFP) images.

Recent studies that evaluated both DENSE and FT in the settings of acute myocardial infarction and cardiac resynchronization therapy found that DENSE outperformed FT for prognostication (2,3). Studies in phantoms and humans have shown that DENSE provides accuracy and reproducibility equivalent to or better than tagging (10,11) (often considered the reference standard) and that DENSE is reproducible for global and segmental strain (12), whereas FT has poor reproducibility for segmental strain (13). While DENSE provides well-validated and more predictive strain than FT, the time needed to acquire DENSE images may not always be compatible with an efficient clinical workflow.

A strain method with performance similar to DENSE and the efficiency of FT would be ideal. Conventional FT uses endocardial and epicardial contours and optical flow (9) and/or an incompressibility model (14) to compute intramyocardial displacement; however, these models have limitations when applied to two-dimensional (2D) images. Deep learning (DL) has demonstrated better performance for general motion tracking tasks than optical flow (15,16) and has been introduced for cine bSSFP motion estimation (17-20). However, to date, DL-based motion estimation for bSSFP has used either unsupervised learning (17, 19, 20) or supervised learning with sub-optimal training data (18), providing suboptimal results. As DENSE provides both myocardial contours and accurate intramyocardial tissue displacement measurements, this disclosure investigated the use of DENSE data to train a supervised deep network (StrainNet) for intramyocardial tissue motion prediction from contour motion. As multiphase endocardial and epicardial contours segmented from DENSE and bSSFP images reflect the same underlying cardiac motion, the results showed that the model trained using DENSE contours could be applied to contours from bSSFP. This model, trained to predict intramyocardial displacement from contour data, is different than previously developed DL models for automatic segmentation and phase unwrapping of DENSE images (21).

Eight centers participated in the study (University of Virginia, Charlottesville; University Hospital, Saint-Etienne, France; University of Kentucky, Lexington; University of Glasgow, Scotland; St Francis Hospital, New York; the Royal Brompton Hospital, London, England; Emory University, Atlanta, Georgia; and Stanford University, Palo Alto, California). Between August 2008 and January 2022, 324 individuals underwent cardiac MRI with DENSE and bSSFP, including 144 healthy volunteers and 180 patients with various types of heart disease. Nineteen individuals with electrocardiographic mistriggering for cine or DENSE imaging, coverage of less than 60% of the cardiac cycle for DENSE, or poor DENSE image quality were excluded (FIG. 1B). Eligible patient groups included those with (a) myocardial infarction (n=62), (b) heart failure with left bundle branch block (n=47), (c) hypertrophic cardiomyopathy (n=17), (d) amyloidosis (n=13), (e) dilated cardiomyopathy (n=10), (f) ischemic heart disease without infarction (n=7), and (g) myocarditis (n=5). Of the 144 healthy volunteers, 45 of them were children and adolescents (mean age, 12 years±3 [SD]). Subsets of this study sample (96 patients and 105 volunteers) were previously reported (2, 3, 12, 22). The prior studies where subsets were reported did not train a DL model to predict intramyocardial displacement from contour data.

In total, 196 data sets were acquired using 3.0-T scanners (MAGNETOM Prisma, Skyra, or Trio; Siemens Healthcare), and 109 were acquired using 1.5-T scanners (MAGNETOM Aera or Avanto; Siemens Healthcare). For each individual, short-axis cine bSSFP images were acquired during repeated breath holds covering the left ventricle (LV) (field of view, 320×320 to 380× 380 mm2; temporal resolution, 30-55 msec, depending on heart rate). Three to four short-axis cine DENSE sections were also acquired (field of view, 200×200 to 360× 360 mm2; temporal resolution, 30-34 msec, resulting in 18-43 frames across the cardiac cycle). Detailed bSSFP and DENSE protocols are provided in the figures.

DENSE images were analyzed by one of five different investigators using previously described methods (23, 24). For bSSFP images, the endocardial and epicardial contours were automatically detected and FT was performed using commercial software (suiteHeart version 5.0.4; NeoSoft). Segmental analyses used the American Heart Association 16-segment model (25).

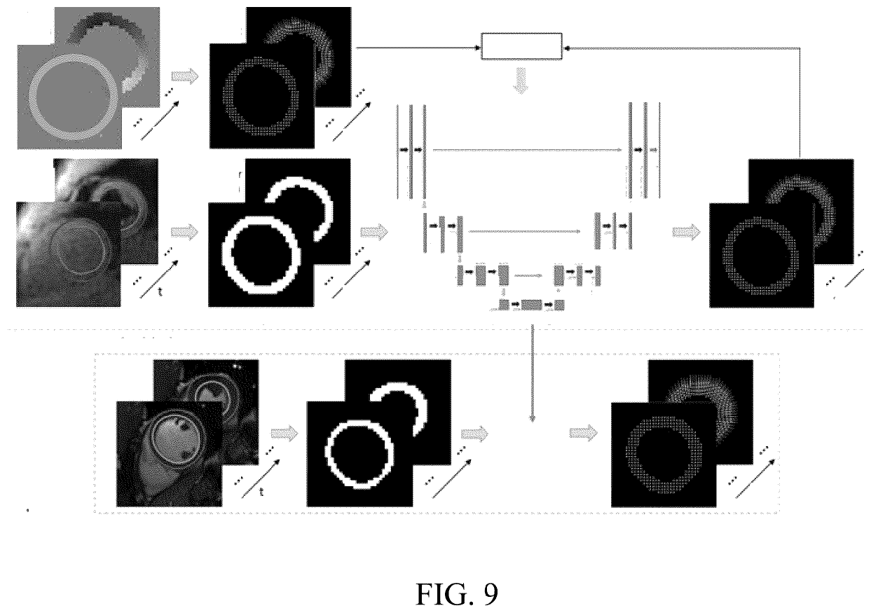

As shown in FIG. 9, a three-dimensional (3D) (2D+time) U-Net (26) was trained to predict intramyocardial displacement, a continuous variable, from contour data. The network depth was three, and the numbers of filters were 64, 128, 256, and 512, from the top to bottom levels, respectively. The kernel sizes were 3×3×3 for the convolutional layers and 4×4×2 for the pooling layers. During training, the U-Net inputs were a time series of myocardial contours derived from DENSE magnitude images, and the ground truth output data were Lagrangian displacement measurements derived from DENSE phase images. For testing, StrainNet was applied to contours derived from FT software applied to standard bSSFP images, and DENSE at matched section locations served as the reference (FIG. 9). Lagrangian strain was computed from StrainNet displacements using the same methods as used for DENSE.

After LV segmentation, images were binarized by filling the myocardial area with ones and the non-myocardial area with zeroes. DENSE images were cropped to a fixed size (48×48) that included the LV. Cine images were scaled to match the mean spatial resolution of DENSE (pixel size, 2.73×2.73 mm2). Morphologic dilation was applied to the bSSFP binary mask to approximately match the wall thickness of bSSFP cine MRI and DENSE, and then the mask images were cropped to the same size as DENSE training data. Data augmentation on the fly was performed by random clockwise and counterclockwise 90° and 180° rotations. The input data for the network were a series of binarized images of LV myocardium of size Nx×Ny×Nt, with Nt representing the number of temporal frames. Outputs were the corresponding Lagrangian displacement maps of size 2×Nx×Ny×Nt, with the factor of 2 accounting for 2D displacement.

Accuracy between StrainNet-estimated displacement from DENSE contours and DENSE ground truth displacements was assessed using the end-point error (EPE), defined by the following equation:

$$EPE = \frac{1}{n} {\sum }_{i = 1}^{n} \sqrt{{( {d}_{est}^{x} ( t ) - {d}_{gt}^{x} ( t ) )}^{2} + {( {d}_{est}^{y} ( t ) - {d}_{gt}^{y} ( t ) )}^{2}} .$$

Average EPE within the myocardial region for the whole time series was calculated, where d_{gt}^{x}(t) and d_{gt}^{y}(t) are the ground truth displacements from DENSE for the x and y directions at time frame t, respectively, dₑₛₜ^{x}(t) and dₑₛₜ^{y}(t) are the StrainNet-estimated displacements, and n is the number of frames.

The 305 patients and controls were randomly divided 80:20 into training and independent testing data sets, and this ratio was applied to each subgroup (healthy adults, healthy children and adolescents, and each disease type). Basal, midlevel, and apical sections were analyzed, resulting in 670 sections from 243 individuals for training and 190 sections from 62 individuals for testing. Fivefold cross-validation was applied within the training data sets, and a single final model was trained using all training data with the optimal hyperparameters selected by cross-validation.

End-systolic global and segmental circumferential strain (Ecc) computed by StrainNet and FT were compared with DENSE using the intraclass correlation coefficient (ICC), Pearson correlation coefficient, coefficient of variation, and Bland-Altman analysis. Transmural strain differences among the subendocardium, middle layer, and subepicardium and strain differences among StrainNet, FT, and DENSE for each segment were assessed by linear mixed-effects models. P value less than 0.05 (two-sided) was considered statistically significant. Statistical analyses were performed (Y.W.) using MATLAB R2018b (Mathworks).

Demographic information and LV volumetric data for patients (mean age, 61 years±14) and healthy controls (99 adults: mean age, 35 years±15; 45 children and adolescents: mean age, 12 years±3) are summarized in Table 1. Males were more common in the patient cohort than in the control cohort (training data set: 67% vs 40%, P<0.001; independent testing data set: 69% vs 62%, P=0.61). Patients with heart disease were older (P<0.001 for both training and testing data sets) and heavier (P<0.001 for training data set, P=0.12 for testing data set) than control adults and had lower LV ejection fraction (P<0.001 for training data set, P=0.28 for testing data set). Also, patients had higher LV end-diastolic volume (P=0.003 for training data set, P=0.23 for testing data set) and LV end-systolic volume (P<0.001 for training data set, P=0.15 for testing data set) compared with control volunteers.

StrainNet training and testing were performed with PyTorch on a 24 GB GPU server (Quadro RTX 6000; NVIDIA). The training time for StrainNet using 670 sections was 251 minutes for 120 epochs. In the test phase, displacement estimation for one entire cardiac cycle per section was accomplished in less than 0.1 second.

Examples of end-systolic displacement and Ecc maps from StrainNet processing of bSSFP image contours and from DENSE for a healthy control and a patient with heart failure with left bundle branch block are shown in FIG. 2 (all cardiac phases are shown in Movies 1 and 2). For the healthy control, qualitatively, StrainNet depicted normal displacement and Ecc, showing good agreement with DENSE. For the patient with heart failure, StrainNet showed simultaneous stretching of the septal segments and contraction of the lateral wall, also showing generally good agreement with DENSE.

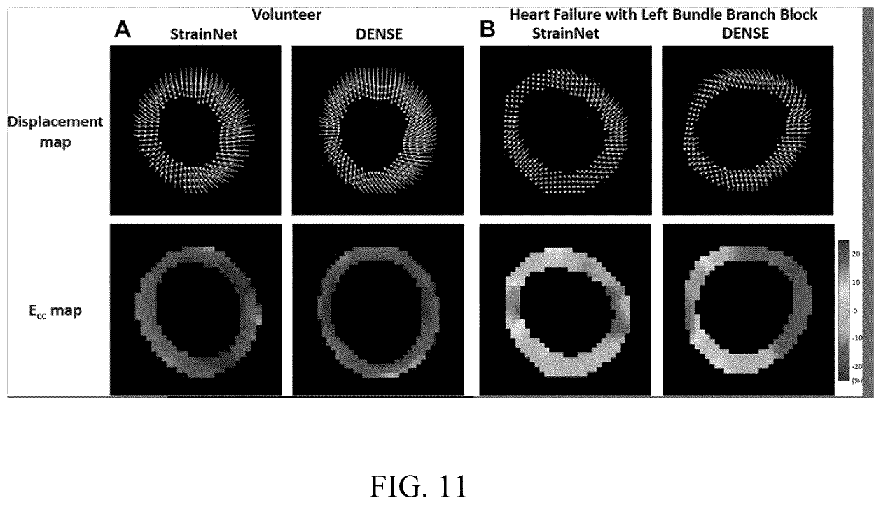

FIG. 11 shows example segmental strain-time curves for a healthy volunteer and a patient with myocardial infarction computed using FT, StrainNet processing of standard cine images, and DENSE. For the healthy volunteer, segmental strain-time curves computed using StrainNet showed better agreement than FT with DENSE for qualitative measurements. FT showed more variability, both spatially and temporally, than StrainNet and DENSE. For the patient with myocardial infarction, both DENSE and StrainNet showed diminished circumferential shortening in the anteroseptal and inferoseptal segments and normal strain elsewhere, whereas FT showed the least amount of circumferential shortening in the anterior and inferior segments, in disagreement with the reference standard DENSE method.

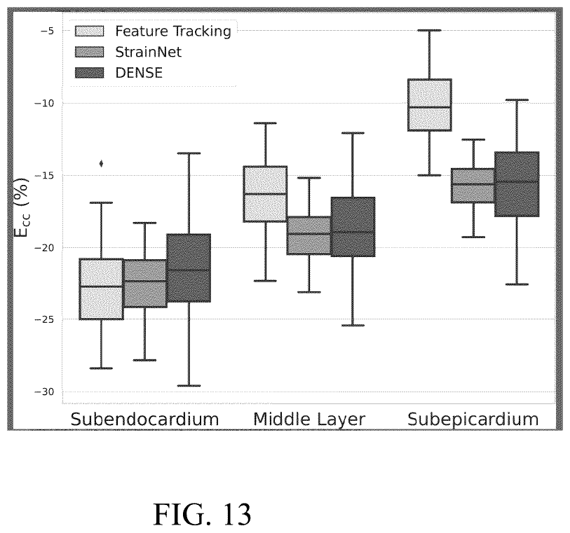

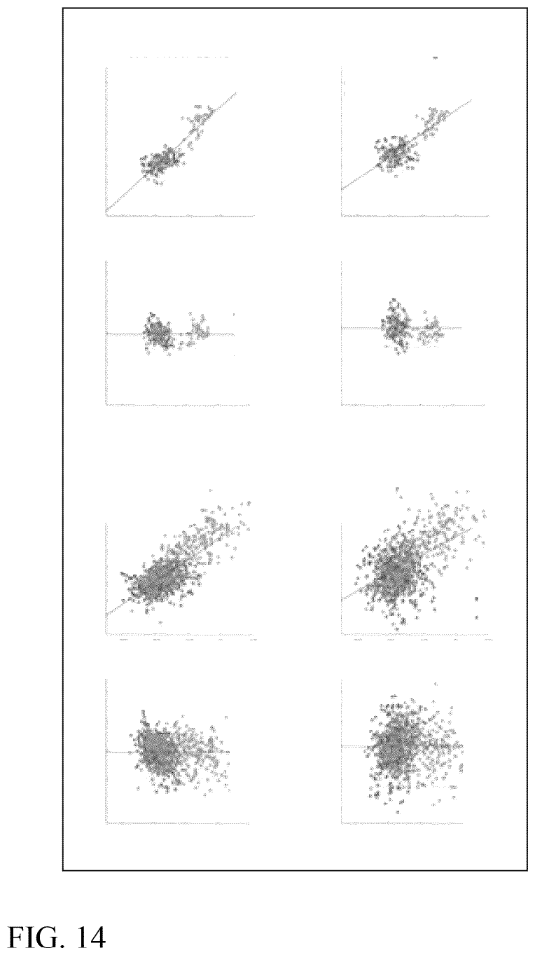

FIG. 13 shows linear correlations and Bland-Altman plots comparing end-systolic Ecc computed using StrainNet and DENSE and FT and DENSE for global and segmental Ecc from all three section positions. Fifty-nine of the 62 individuals in the independent test set with images from basal, midlevel, and apical sections were included. This disclosure found no evidence of differences between StrainNet and DENSE for end-systolic global Ecc (P=0.42) and segmental Ecc (P=0.08), with corresponding biases of −0.51% (95% limits of agreement: −5.74%, 4.72%) and −0.55% (95% limits of agreement: −9.16%, 8.07%), respectively; whereas there were significant differences between FT and DENSE for global Ecc (P=0.003) and segmental Ecc (P<0.001), with corresponding biases of 1.6% (95% limits of agreement: −4.97%, 8.09%) and 1.5% (−12.2%, 15.3%), respectively.

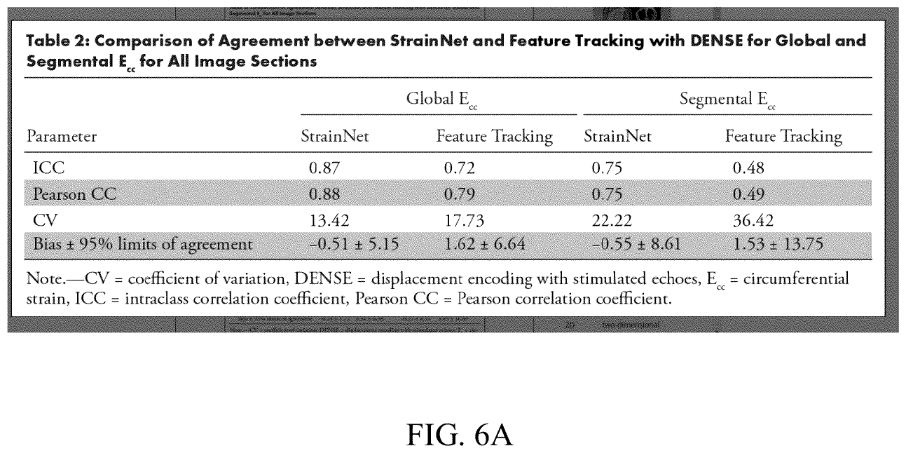

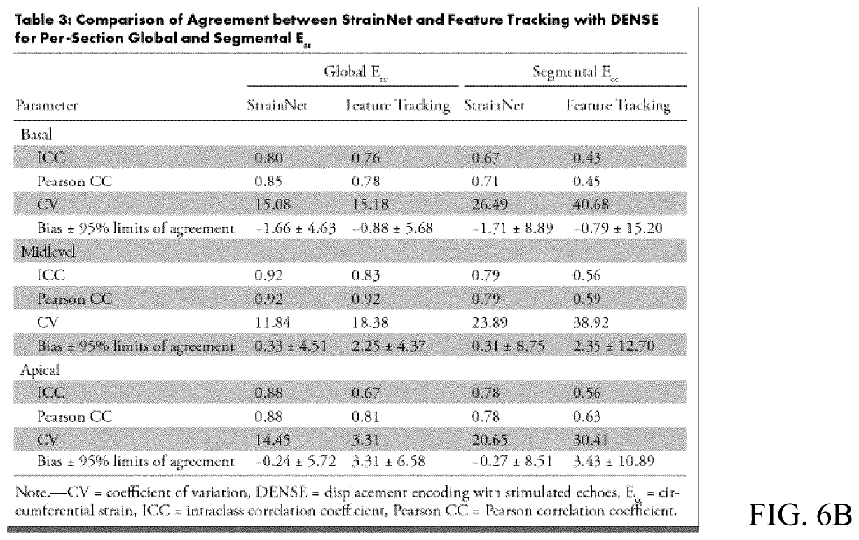

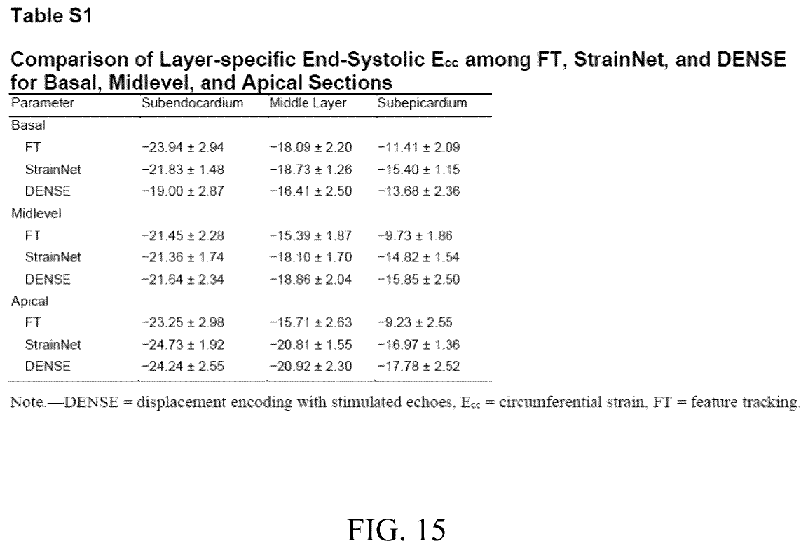

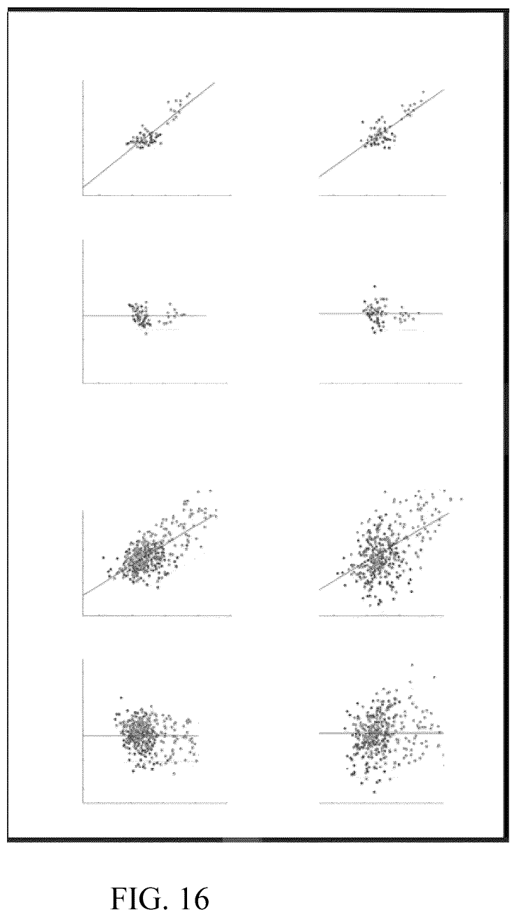

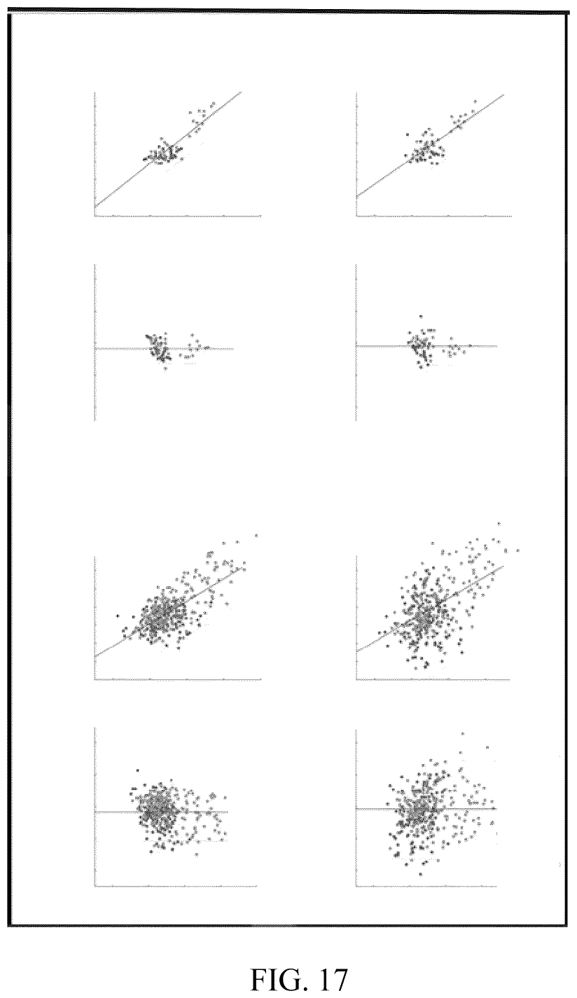

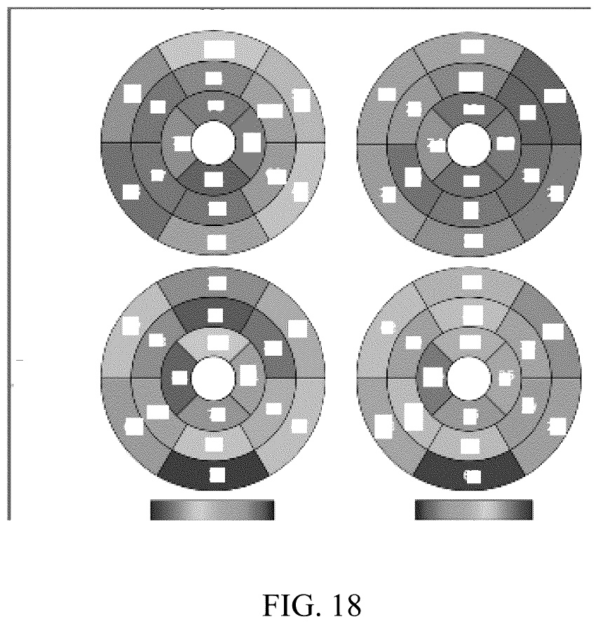

Table 2 of FIG. 6A summarizes ICC, Pearson correlation coefficient, coefficient of variation, and the corresponding biases and limits of agreement results comparing FT and StrainNet with DENSE for the assessment of global and segmental Ecc. The ICCs between StrainNet and DENSE and FT and DENSE were 0.87 versus 0.72, respectively, for global Ecc and 0.75 versus 0.48, respectively, for segmental Ecc, showing that StrainNet had good agreement with DENSE and outperformed FT for both global and segmental Ecc. Table 3 and FIGS. 15-17 summarize per-section quantitative results for global and segmental Ecc for basal, midlevel, and apical sections, respectively, which showed that StrainNet had better correlation and less variation from DENSE compared with FT for basal, midlevel, and apical sections and smaller biases for mid and apical sections. Bull's-eye plots summarize per-segment ICC, coefficient of variation, bias, and 95% limits of agreement between StrainNet and DENSE and between FT and DENSE (FIG. 18). StrainNet showed better agreement with DENSE and less bias for all metrics.

While strain-dedicated methods such as DENSE provide the most accurate assessment of global and segmental strain, it may not always be practical to add strain-dedicated acquisitions to the cardiac MRI protocol in busy clinical settings. The ability to perform strain analysis of routine bSSFP cine images supports an efficient clinical workflow, and DL models trained on strain-dedicated data may provide a means to outperform conventional FT for this task. This study leveraged DENSE data from more than 300 patients with heart disease and healthy controls to develop a DL framework that can predict intramyocardial displacement from myocardial contours and showed that the resulting 3D U-Net, named StrainNet, can be successfully used for strain analysis of standard cine images. There are several major findings of this study. First, for both healthy volunteers and patients, StrainNet predicted intramyocardial displacement and strain from myocardial contour motion and showed good agreement with DENSE. Second, for the analysis of routine cine MRI, StrainNet showed better agreement than FT with DENSE for global and segmental Ecc. Additional conclusions are the following: (a) StrainNet was effective when applied to contours from either DENSE images or cine images, (b) StrainNet showed good reliability for segmental Ecc, with an ICC greater than 0.75 for midventricular and apical sections, and (c) transmural layer-specific Ecc measurements by using StrainNet were more consistent than FT with DENSE.

Learning-based myocardial motion tracking methods have been investigated previously (17-20) and demonstrated the potential for high performance by leveraging big data and the ability of convolutional neural networks to extract intangible and multiscale features. The main challenge in the development of motion-tracking methods by using DL is the lack of ground truth data (18). Previous studies have shown the feasibility of regional strain assessment by DL using cine MRI, but they used classic optical flow to generate the motion ground truth (18) or unsupervised loss functions during training (17, 19, 20). This study benefitted from the availability of multicenter DENSE data sets from 305 individuals with healthy hearts and with various types of heart diseases and from the strong correspondence between DENSE and standard cine images in the depiction of heart motion, allowing us to generate accurate displacements, global Ecc, and segmental Ecc, from routine cine MRI. StrainNet performance benefited from the diversity of the data, which included multiple diseases, multiple data acquisition sites, multiple observers for data postprocessing, a wide range of ages (adults, adolescents, and children), and different magnetic field strengths (1.5 T and 3.0 T). As cine bSSFP and cine DENSE generally have similar temporal resolutions, this study found that no additional manipulation was needed to handle variability in temporal resolution or number of cardiac phases. Compared with previous DL-based cardiac motion-tracking networks, our supervised learning framework took advantage of the accuracy of DENSE, resulting in a lower average EPE of 0.75 mm±0.35 compared with previously reported DL-based motion-tracking results of 2.9 mm±1.5 (17) and 0.94 mm±1.59 (18). With respect to the spatial resolution matching between bSSFP cine MRI and cine DENSE, another potential approach would be to interpolate DENSE training data to a higher spatial resolution. However, to avoid altering the ground truth DENSE displacement data, binarized bSSFP cine images were under-sampled to match the DENSE resolution.

For breath-hold, segmented DENSE acquisitions (27), uncertainty in the measurement of displacement and strain arises from noise, imperfect breath holding (28), variability in prescribing the scan plane, variability in physiology that may occur over time, and variability in generating endocardial and epicardial contours. These factors have been investigated previously, and the reproducibility of DENSE has been shown to be outstanding compared with other myocardial strain imaging methods (12, 29). For StrainNet, uncertainty in the measurement of displacement and strain is due to variability in prescribing the scan plane, variability in physiology that may occur over time, and differences that may occur in contour generation. While these factors and strain reproducibility have been studied previously for DENSE, future research with StrainNet should investigate and quantify intra-user, interuser, and intersession variability in displacement and strain quantification. Presently neither StrainNet nor FT shows good agreement with DENSE for radial strain (21), thus more development is needed before comparing radial strain results generated by the various methods.

In conclusion, using data from healthy adult and pediatric volunteers and adults with various types of heart disease, a DL framework to accurately predict intramyocardial motion from contour motion was developed. Its application to standard cine MRI showed better agreement than FT for the quantification of global and segmental Ecc, using DENSE as the reference standard. This approach enables strain analysis of routine cine MRI with accuracy similar to that of strain-dedicated DENSE. StrainNet may facilitate greater use of strain cardiac MRI in research and in the clinical setting. In the future, StrainNet could likely be improved by further increasing the size and diversity of the training data set and retraining the network. With the sound framework of StrainNet applied to contoured images of the heart proved by the current work, this disclosure will allow for investigating the application of the network to other modalities such as cardiac CT and echocardiography cine images. Along these lines, StrainNet provides the potential to standardize strain values across different imaging modalities. Additional FIGS. 15-17 attached to this disclosure show full details of cine and displacement encoding with stimulated echoes with imaging parameters. For each sample, short-axis cine balanced steady-state free precession images were acquired during repeated breath holds covering the whole left ventricle. Typical imaging parameters were as follows: repetition time, 3.0-3.4 msec; echo time, 1.5-1.7 msec; matrix size, 192×224-224×256; field of view, 320×320-380×380 mm2; and temporal resolution, 30-55 msec, depending on heart rate. For each sample, 3-4 short-axis cine displacement encoding with stimulated echoes (DENSE) sections were also acquired. Typical DENSE imaging parameters were as follows: repetition time, 15-17 msec; echo time, 1.06-1.26 msec; pixel size, 1.56×1.56-3.13×3.13 mm2; matrix size, 64×64-128×128; field of view 200×200-360× 360 mm2; temporal resolution, 30-34 msec (resulting in 18-43 frames across the cardiac cycle); region of signal generation, 120×120-360×360 mm2; one-dimensional or two-dimensional in-plane displacement encoding using the simple three-point method; displacement-encoding frequency, 0.1 cycles/mm; ramped flip angle with final flip angle of 15°; fat suppression; and four to six spiral interleaves per image, with two interleaves acquired per heartbeat. Each cine DENSE acquisition was performed during end-expiratory breath holding over 14 cardiac cycles.

Embodiments of this disclosure include a computer implemented method of measuring intramyocardial tissue displacement with image data, the method includes retrieving a medical image of a subject with a magnetic resonance imaging (MRI) system, having at least one processor, wherein the MRI system retrieves the medical image with a balanced steady-state free precession (bSSFP) pulse sequence. The method includes determining, by the processor, intramyocardial tissue displacement within the medical image by using a neural network, wherein the neural network has been trained with training data calculated from Displacement-ENcoding with Stimulated Echoes (DENSE) image data. Training the neural network includes using a training computer to perform computerized training steps including:
(i) generating a set of contour motion image data from magnitude data from the DENSE image data;
(ii) using the neural network to calculate estimated displacement image data from the contour motion image data;
(iii) generating a set of ground truth displacement image data with phase data from the DENSE image data;
(iv) calculating error data by comparing the estimated displacement image data with the ground truth displacement image data; and
(v) updating parameters of the neural network with the error data.

In some embodiments, retrieving a medical image of a subject includes retrieving a two dimensional (2D) bSSFP medical image of the subject. In some embodiments, retrieving the medical image comprises retrieving cine image data with the MRI system using the bSSFP pulse.

The method incorporates using a processor to generate frames of test image magnitude data from the cine image data and segment the test image magnitude data to track pixels of the medical image corresponding to endocardial contours and epicardial contours of a myocardium of the subject. Applying morphological dilation to a bSSFP binary mask binarizes segmented test image magnitude data to generate test contour motion data for the medical image. Under sampling the segmented test image magnitude data generates the test contour motion data with spatial resolution matching between the DENSE image data and the test contour motion data.

In some embodiments, this disclosure uses the test contour motion data as an input to the neural network to calculate intramyocardial tissue displacement from the cine image data. Other embodiments include using a convolutional neural network as the neural network. A 3D U-Net neural network may be used as the convolutional neural network.

A system according to this disclosure includes a processor and a memory having instructions stored thereon to calculate intramyocardial displacement in medical image scans, wherein execution of the instructions by the processor causes the processor to retrieve a medical image of a subject with a magnetic resonance imaging (MRI) system in communication with the processor, wherein the MRI system retrieves the medical image with a balanced steady-state free precession (bSSFP) pulse sequence. The system determines, with the processor, intramyocardial tissue displacement within the medical image by using a neural network, wherein the neural network has been trained with training data calculated from Displacement-ENcoding with Stimulated Echoes (DENSE) image data. The instructions in the memory implement a neural network to calculate the intramyocardial displacement. The neural network may be a previously trained neural network trained by a training computer to perform computerized steps comprising:
(i) generating a set of contour motion image data from magnitude data from the DENSE image data;
(ii) using the neural network to calculate estimated displacement image data from the contour motion image data;
(iii) generating a set of ground truth displacement image data with phase data from the DENSE image data;
(iv) calculating error data by comparing the estimated displacement image data with the ground truth displacement image data; and
(v) updating parameters of the neural network with the error data.

In another embodiment, a non-transitory computer readable medium has instructions stored thereon to calculate intramyocardial displacement in medical image scans, wherein execution of the instructions by a computer with a processor causes the computer to retrieve a medical image of a subject with a magnetic resonance imaging (MRI) system in communication with the processor, wherein the MRI system retrieves the medical image with a balanced steady-state free precession (bSSFP) pulse sequence. The instructions further allow for the computer and its processor to determine intramyocardial tissue displacement within the medical image by using a neural network, wherein the neural network has been trained with training data calculated from Displacement-ENcoding with Stimulated Echoes (DENSE) image data.

### LIST OF REFERENCES

1. Giusca S, Korosoglou G, Montenbruck M, et al. Multiparametric early detection and prediction of cardiotoxicity using myocardial strain, T1 and T2 mapping, and biochemical markers: a longitudinal cardiac resonance imaging study during 2 years of follow-up. Circ Cardiovasc Imaging 2021; 14(6):e012459. Crossref, Medline, Google Scholar
2. Bilchick K C, Auger D A, Abdishektaei M, et al. CMR DENSE and the Seattle Heart Failure Model inform survival and arrhythmia risk after CRT. JACC Cardiovasc Imaging 2020; 13(4):924-936. Crossref, Medline, Google Scholar
3. Mangion K, Carrick D, Carberry J, et al. Circumferential strain predicts major adverse cardiovascular events following an acute ST-segment-elevation myocardial infarction. Radiology 2019; 290(2):329-337. Link, Google Scholar
4. Ernande L, Thibault H, Bergerot C, et al. Systolic myocardial dysfunction in patients with type 2 diabetes mellitus: identification at MR imaging with cine displacement encoding with stimulated echoes. Radiology 2012; 265(2):402-409. Link, Google Scholar
5. Axel L, Dougherty L. Heart wall motion: improved method of spatial modulation of magnetization for MR imaging. Radiology 1989; 172(2):349-350. Link, Google Scholar
6. Aletras A H, Ding S, Balaban R S, Wen H. DENSE: displacement encoding with stimulated echoes in cardiac functional MRI. J Magn Reson 1999; 137(1):247-252. Crossref, Medline, Google Scholar
7. Kim D, Gilson W D, Kramer C M, Epstein F H. Myocardial tissue tracking with two-dimensional cine displacement-encoded MR imaging: development and initial evaluation. Radiology 2004; 230(3):862-871. Link, Google Scholar
8. Osman N F, Sampath S, Atalar E, Prince J L. Imaging longitudinal cardiac strain on short-axis images using strain-encoded MRI. Magn Reson Med 2001; 46(2):324-334. Crossref, Medline, Google Scholar
9. Hor K N, Baumann R, Pedrizzetti G, et al. Magnetic resonance derived myocardial strain assessment using feature tracking. J Vis Exp 2011; (48):2356. Medline, Google Scholar
10. Young A A, Li B, Kirton R S, Cowan B R. Generalized spatiotemporal myocardial strain analysis for DENSE and SPAMM imaging. Magn Reson Med 2012; 67(6): 1590-1599. Crossref, Medline, Google Scholar
11. Kar J, Knutsen A K, Cupps B P, Pasque M K. A validation of two-dimensional in vivo regional strain computed from displacement encoding with stimulated echoes (DENSE), in reference to tagged magnetic resonance imaging and studies in repeatability. Ann Biomed Eng 2014; 42(3):541-554. Crossref, Medline, Google Scholar
12. Auger D A, Ghadimi S, Cai X, et al. Reproducibility of global and segmental myocardial strain using cine DENSE at 3 T: a multicenter cardiovascular magnetic resonance study in healthy subjects and patients with heart disease. J Cardiovasc Magn Reson 2022; 24(1):23. Crossref, Medline, Google Scholar
13. Mangion K, Burke N M M, McComb C, Carrick D, Woodward R, Berry C. Feature-tracking myocardial strain in healthy adults—a magnetic resonance study at 3.0 tesla. Sci Rep 2019; 9(1):3239. Crossref, Medline, Google Scholar
14. Bistoquet A, Oshinski J, Skrinjar O. Myocardial deformation recovery from cine MRI using a nearly incompressible biventricular model. Med Image Anal 2008; 12(1):69-85. Crossref, Medline, Google Scholar
15. Ilg E, Mayer N, Saikia T, Keuper M, Dosovitskiy A, Brox T. FlowNet 2.0: Evolution of Optical Flow Estimation with Deep Networks. Proc Cvpr Ieee, 2017; 1647-1655. Google Scholar
16. Sun D Q, Yang X D, Liu M Y, Kautz J. PWC-Net: CNNs for optical flow using pyramid, warping, and cost volume. 2018 Ieee/Cvf Conference on Computer Vision and Pattern Recognition (Cvpr), 2018; 8934-8943. Crossref, Google Scholar
17. Morales M A, van den Boomen M, Nguyen C, et al. DeepStrain: a deep learning workflow for the automated characterization of cardiac mechanics. Front Cardiovasc Med 2021; 8:730316. Crossref, Medline, Google Scholar
18. Qiao M, Wang Y, Guo Y, Huang L, Xia L, Tao Q. Temporally coherent cardiac motion tracking from cine MRI: Traditional registration method and modern CNN method. Med Phys 2020; 47(9):4189-4198. Crossref, Medline, Google Scholar
19. Yu H C, Sun S H, Yu H C, et al. FOAL: Fast Online Adaptive Learning for cardiac motion estimation. 2020 Ieee/Cvf Conference on Computer Vision and Pattern Recognition (Cvpr), 2020:4312-4322. Crossref, Google Scholar
20. Qin C, Bai W J, Schlemper J, et al. Joint motion estimation and segmentation from undersampled cardiac MR Image. In: Knoll F, Maier A, Rueckert D, eds. Machine learning for medical image reconstruction. MLMIR 2018. Lecture notes in computer science, vol 11074. Springer, 2018; 55-63. Crossref, Google Scholar
21. Ghadimi S, Auger D A, Feng X, et al. Fully-automated global and segmental strain analysis of DENSE cardiovascular magnetic resonance using deep learning for segmentation and phase unwrapping. J Cardiovasc Magn Reson 2021; 23(1):20. Crossref, Medline, Google Scholar
22. Jing L, Binkley C M, Suever J D, et al. Cardiac remodeling and dysfunction in childhood obesity: a cardiovascular magnetic resonance study. J Cardiovasc Magn Reson 2016; 18(1):28. Crossref, Medline, Google Scholar
23. Spottiswoode B S, Zhong X, Lorenz C H, Mayosi B M, Meintjes E M, Epstein F H. Motion-guided segmentation for cine DENSE MRI. Med Image Anal 2009; 13(1):105-115. Crossref, Medline, Google Scholar
24. Spottiswoode B S, Zhong X, Hess A T, et al. Tracking myocardial motion from cine DENSE images using spatiotemporal phase unwrapping and temporal fitting. IEEE Trans Med Imaging 2007; 26(1): 15-30. Crossref, Medline, Google Scholar
25. Cerqueira M D, Weissman N J, Dilsizian V, et al. Standardized myocardial segmentation and nomenclature for tomographic imaging of the heart. A statement for healthcare professionals from the Cardiac Imaging Committee of the Council on Clinical Cardiology of the American Heart Association. Circulation 2002; 105(4):539-542. Crossref, Medline, Google Scholar
26. Ronneberger O, Fischer P, Brox T. U-Net: convolutional networks for biomedical image segmentation. In: Navab N, Hornegger J, Wells W, Frangi A, eds. Medical image computing and computer-assisted intervention—MICCAI 2015. MICCAI 2015. Lecture Notes in Computer Science, vol 9351. Springer, 2015; 234-241. Crossref, Google Scholar
27. Zhong X, Spottiswoode B S, Meyer C H, Kramer C M, Epstein F H. Imaging three-dimensional myocardial mechanics using navigator-gated volumetric spiral cine DENSE MRI. Magn Reson Med 2010; 64(4): 1089-1097. Crossref, Medline, Google Scholar
28. Abdi M, Bilchick K C, Epstein F H. Compensation for respiratory motion-induced signal loss and phase corruption in free-breathing self-navigated cine DENSE using deep learning. Magn Reson Med 2023; 89(5):1975-1989. Crossref, Medline, Google Scholar
29. Lin K, Meng L, Collins J D, Chowdhary V, Markl M, Carr J C. Reproducibility of cine displacement encoding with stimulated echoes (DENSE) in human subjects. Magn Reson Imaging 2017; 35:148-153. Crossref, Medline, Google Scholar

1. A computer implemented method of measuring intramyocardial tissue displacement with image data, the method comprising
retrieving a medical image of a subject with a magnetic resonance imaging (MRI) system, having at least one processor, wherein the MRI system retrieves the medical image with a balanced steady-state free precession (bSSFP) pulse sequence; and
determining, by the processor, intramyocardial tissue displacement within the medical image by using a neural network, wherein the neural network has been trained with training data calculated from Displacement-ENcoding with Stimulated Echoes (DENSE) image data.

2. The computer implemented method of claim 1, wherein training the neural network comprises using a training computer to perform computerized steps comprising:
(vi) generating a set of contour motion image data from magnitude data from the DENSE image data;
(vii) using the neural network to calculate estimated displacement image data from the contour motion image data;
(viii) generating a set of ground truth displacement image data with phase data from the DENSE image data;
(ix) calculating error data by comparing the estimated displacement image data with the ground truth displacement image data; and
(x) updating parameters of the neural network with the error data.

3. The computer implemented method of claim 1, wherein retrieving a medical image of a subject comprises retrieving a two dimensional (2D) bSSFP medical image of the subject.

4. The computer implemented method of claim 1, wherein retrieving the medical image comprises retrieving cine image data with the MRI system using the bSSFP pulse.

5. The computer implemented method of claim 4, further comprising using the processor to generate frames of test image magnitude data from the cine image data and segment the test image magnitude data to track pixels of the medical image corresponding to endocardial contours and epicardial contours of a myocardium of the subject.

6. The computer implemented method of claim 5, further comprising applying morphological dilation to a bSSFP binary mask to binarize segmented test image magnitude data to generate test contour motion data for the medical image.

7. The computer implemented method of claim 6, further comprising under sampling the segmented test image magnitude data to generate the test contour motion data with spatial resolution matching between the DENSE image data and the test contour motion data.

8. The computer implemented method of claim 6, further comprising using the test contour motion data as an input to the neural network to calculate intramyocardial tissue displacement from the cine image data.

9. The computer implemented method of claim 1, further comprising using a convolutional neural network as the neural network.

10. The computer implemented method of claim 9, further comprising using a 3D U-Net neural network as the convolutional neural network.

11. A system comprising:
a processor; and
a memory having instructions stored thereon to calculate intramyocardial displacement in medical image scans, wherein execution of the instructions by the processor causes the processor to:
retrieve a medical image of a subject with a magnetic resonance imaging (MRI) system in communication with the processor, wherein the MRI system retrieves the medical image with a balanced steady-state free precession (bSSFP) pulse sequence; and
determining, by the processor, intramyocardial tissue displacement within the medical image by using a neural network, wherein the neural network has been trained with training data calculated from Displacement-ENcoding with Stimulated Echoes (DENSE) image data.

12. The system of claim 12, wherein the instructions in the memory implement a neural network to calculate the intramyocardial displacement.

13. The system of claim 12, wherein the neural network is a previously trained neural network trained by a training computer to perform computerized steps comprising:
(i) generating a set of contour motion image data from magnitude data from the DENSE image data;
(ii) using the neural network to calculate estimated displacement image data from the contour motion image data;
(iii) generating a set of ground truth displacement image data with phase data from the DENSE image data;
(iv) calculating error data by comparing the estimated displacement image data with the ground truth displacement image data; and
(v) updating parameters of the neural network with the error data.

14. A non-transitory computer readable medium having instructions stored thereon to calculate intramyocardial displacement in medical image scans, wherein execution of the instructions by a computer with a processor causes the computer to:
retrieve a medical image of a subject with a magnetic resonance imaging (MRI) system in communication with the processor, wherein the MRI system retrieves the medical image with a balanced steady-state free precession (bSSFP) pulse sequence; and
determining, by the processor, intramyocardial tissue displacement within the medical image by using a neural network, wherein the neural network has been trained with training data calculated from Displacement-ENcoding with Stimulated Echoes (DENSE) image data.
